(19) United States
(12) Patent Application Publication
(10) Pub. No.: US 2025/0226266 A1
(43) Pub. Date: Jul. 10, 2025

(54) IN-SITU, PRE-IMPLANT WAFER CHARACTERIZATION SYSTEM AND METHOD

(71) Applicant: Axcelis Technologies, Inc., Beverly, MA (US)

(72) Inventor: Scott Edwin Galica, Chelmsford, MA (US)

(21) Appl. No.: 18/991,789

(22) Filed: Dec. 23, 2024

Related U.S. Application Data
(60) Provisional application No. 63/619,339, filed on Jan. 10, 2024.

(51) Int. Cl. H01L 21/66; H01J 37/20; H01J 37/22; H01J 37/317; H01L 21/67

(52) U.S. Cl. CPC H01L 22/12; H01J 37/20; H01J 37/22; H01J 37/3171; H01L 21/67288

(57) ABSTRACT

An ion implantation system includes a wafer inspection system for inspecting wafers prior to ion implantation. The inspection facilitates diagnostics by helping distinguish the performance of an ion implantation process from the performance of upstream processes that affect the ion implantation process. The inspection may take place while the wafer is on an aligner, while it is in a load lock chamber, or while it is otherwise being processed by a wafer transport system in an end station. The inspection may be carried out without adding delay to wafer processing. The inspection may include modulated optical resonance (MOR) spectroscopy, and the inspection may be carried out through an optical fiber. The optical circuit may include a wavelength coupler so that a pump laser and probe laser of the MOR system can focus through one lens on a narrowly determined inspection point.

### REFERENCE TO RELATED APPLICATION

This Application claims the benefit of U.S. Provisional Application No. 63/619,339, filed on Jan. 10, 2024, the contents of which are incorporated by reference in their entirety.

### BACKGROUND

Ion implantation systems are used in the integrated circuit (IC) device manufacturing industry for damage engineering and to provide semiconductor wafers with controlled doses of impurities. A mask may be used to define a pattern for an implant. Such factors as the depth and concentration profile of the implant may be varied by selecting operating parameters for the ion implantation system. Manufacturing an IC device involves many ion implantations having various patterns, different types of impurities, varying concentrations, and various depths.

### BRIEF DESCRIPTION OF THE DRAWINGS

FIG. 1 illustrates an end station for an ion implantation system in accordance with some aspects of the present disclosure.

FIG. 2 illustrates an aligner in accordance with some embodiments.

FIG. 3 illustrates a wafer inspection system in accordance with some embodiments.

FIG. 4 is another illustration of a wafer inspection system in accordance with some embodiments.

FIG. 5 illustrates an ion implantation system in accordance with some embodiments.

FIG. 6 illustrates an end station for an ion implantation system in accordance with some embodiments.

FIG. 7 illustrates an end station for an ion implantation system in accordance with some embodiments.

FIG. 8 illustrates an end station robot in accordance with some embodiments.

FIG. 9 illustrates an end station for an ion implantation system in accordance with some embodiments.

FIG. 10A-10C illustrate a workpiece handler in accordance with some embodiments.

FIG. 11 provides a flow chart for a method in accordance with some aspects of the present disclosure.

### DETAILED DESCRIPTION

The present disclosure provides many different embodiments, or examples, for implementing different features of this disclosure. Specific components and arrangements are provided to clarify and exemplify the disclosure. These specific examples should not be interpreted as limiting the scope of what is claimed.

One aspect of the present disclosure relates to an ion implantation system that provides an inspection of a wafer prior to performing ion implantation. The success of the ion implantation will depend on the condition of the wafer prior to the ion implantation process. For example, an ion implantation for a shallower depth is typically performed after one or more ion implantations targeting greater depths. The correct dose for the ion implantation targeting the shallower depth depends not only on the desired charge carrier concentration at the shallower depth but also the type and concentration of dopants left by the prior ion implantation processes. Variations in those processes can affect the current process.

Another condition that will affect the current ion implantation process is the crystalline structure of the substrate. The depth of an ion implantation is strongly affected by disorder (amorphism) in the crystalline structure. In some instances, the crystalline structure is made partially amorphous intentionally so as to help control the depth of an implant. In some instances, the crystalline structure becomes partially amorphous incidentally or unintentionally. For all these reasons, the inventors have found that inspections of wafers conducted post ion implantation are often not probative of whether the current ion implantation process is operating as intended or needs adjustment. The present disclosure solves this problem with an ion implantation system that has a built-in wafer inspection system that can provide inspections of wafers immediately prior to ion implantation.

An ion implantation system may include an ion source, a beamline, an end station, and a wafer transport system. The wafer transport system is in the end station and transports wafers from wafer carriers that are at or near atmospheric pressure to a workpiece handler that is under vacuum in a process chamber of the end station. The ion source generates an ion beam, the beamline guides the ion beam, and the workpiece handler holds the wafer in a path of the ion beam. The workpiece handler determines the implant plane, which is a plane in which the ion beam strikes the wafer. In some embodiments, the workpiece handler is configured to move the wafer across the path of the ion beam while holding the wafer on a fixed implant plane.

In some embodiments, the wafer transport system includes a load lock, an aligner, and one or more robots. The aligner rotates wafers to set their orientations with reference to alignment marks provided on the wafers. The aligner is provided because the depth of an ion implantation may vary in relation to an angle of incidence of an ion beam in relation to an orientation of a wafer's crystalline structure. The load lock system is used to transition the wafer from the at or near atmospheric pressure condition outside the process chamber to the vacuum condition within the process chamber. A first robot may pull the wafer from a wafer carrier and place the wafer on the aligner. After the wafer has been aligned, a second robot may remove the wafer from the aligner and place it in the load lock chamber. After the load lock chamber has been evacuated, a third robot may remove the wafer from the load lock chamber and place the wafer on the wafer hander. The workpiece handler may place the wafer on the implant plane and may scan the wafer along the implant plane. Alternatively, the aligner may be in the process chamber.

In some embodiments, the inspection takes place while the wafer is in a load lock chamber. The wafer dwells in the load lock chamber while the load lock chamber is being evacuated. This provides an opportunity to inspect the wafer without adding to process time. In some alternative embodiments, the wafer carriers are transported through the load lock chamber rather than individual wafers and inspection within the load lock chamber may not be practical.

In some embodiments, the inspection takes place while the wafer is on the aligner. The wafer may remain stationary for a brief period while on the aligner. The scan may be carried out during this brief period without adding to the wafer processing time. In some embodiments, the wafer is inspected after alignment. Inspection after alignment provides precise determination of an inspection point on the wafer. In some embodiments, the inspection is closer to a periphery of the wafer than a center of the wafer. Using a point near the periphery as the inspection point facilitates maintaining an output coupler or other detection head of the wafer inspection system at a fixed position that will be near the wafer surface while the wafer is on the aligner. Having the detection head near the periphery facilitates sliding wafers under the detection head as the wafers are loaded onto the aligner. An additional advantage of inspection on the aligner is that the aligner provides a convenient attachment point for parts of the wafer inspection system.

In some embodiments, the inspection is a pointwise inspection. A pointwise inspection has advantages over a complete wafer scan including the ability to complete the inspection quickly, and the ability to conduct the inspection with smaller equipment. In some embodiments, the wafer on the aligner is inspected at a plurality of inspection points that are all equidistant from a center of the wafer. The ability of the aligner to rotate the wafer provides an opportunity to reposition the wafer quickly and precisely so that inspections may be carried out at a plurality of inspection points using one stationary detection head.

In some embodiments, the inspection takes place while the wafer is in the grip of one of the robotic arms of the wafer transport system. A wafer may remain stationary on a robotic arm while waiting for a destination such as the aligner, the load lock chamber, or the workpiece handler, to be cleared. Such pauses provide opportunities to inspect the wafer without increasing wafer processing time.

In some embodiments, the inspection takes place while the wafer is on the workpiece handler. The inspection may take place while the wafer is at a first orientation. The first orientation may be convenient for inspection. The workpiece handler may then reorient the wafer so that its surface is on the implant plane.

In some embodiments the inspection is carried out through an optical fiber. Carrying out the inspection through an optical fiber provides flexibility in positioning other components of the inspection system. In some embodiments, a component of the inspection system is positioned outside the process chamber. In some embodiments, the component is a laser. In some embodiments, the component is a photodetector. Placing components of the inspection system outside the process chamber may prolong their life. In some embodiments, a component of the inspection system is mounted on the aligner. Mounting the component to the aligner provides a short path for an optical fiber extending from the component to an output coupler held in a fixed position over the aligner.

In some embodiments, the inspection system is a modulated optical reflectance (MOR) system. An MOR system includes a pump laser, a probe laser, and a photodetector. In some embodiments, the MOR system includes an optical circuit comprising optical fibers and a wavelength coupler. The wavelength coupler facilitates focusing the pump laser and the probe laser on the same inspection point. The wavelength coupler allows a pump laser output, a probe laser output, and a reflection of the probe laser off the inspection point to all travel through a single optical fiber and a single lens in an output coupler attached to the single optical fiber.

In some embodiments, an output coupler including a lens at the end of the optical fiber is held in a fixed position directly above an inspection point. Maintaining the output coupler in a fixed position eliminates a source of variability in the inspection data. In some embodiments the output coupler is moveable. For example, a robot may be provided to shift the position of the output coupler. The moveable output coupler may facilitate inspecting wafers without interfering with the normal movement of wafers through the end station. The lens may focus the pump laser and probe laser outputs onto the inspection point and may collate a reflection of the probe laser from the inspection point into the optical fiber. In some embodiments, the pump laser is modulated at a single frequency. Using a single frequency for each inspection (as opposed to a frequency scan) allows the inspection to take place quickly and can be sufficiently probative for the purposes of the wafer inspection system.

The wafer inspection system provides inspection data which may be used to make a diagnostic determination concerning a wafer. In some embodiments, the wafer is discarded based on the diagnostic determination. In some embodiments, the wafer is subjected to remedial processing based on the diagnostic determination. The ion implantation may be postponed until the remedial processing has been completed. In some embodiments, the ion implantation carried out after the inspection is modified based on the diagnostic determination. In some embodiments, the operation of a processing tool upstream from the ion implantation system is modified based on the diagnostic determination.

In some embodiments, making the diagnostic determination from the inspection data involves comparing the inspection data to previously acquired inspection data. In some embodiments, the previously acquired inspection data was obtained by the inspection system from inspecting one or more previously processed wafers. In some embodiments, a second inspection of the wafer takes place after the ion implantation and the diagnostic determination is made by comparing the results of the second inspection to the results of the first inspection.

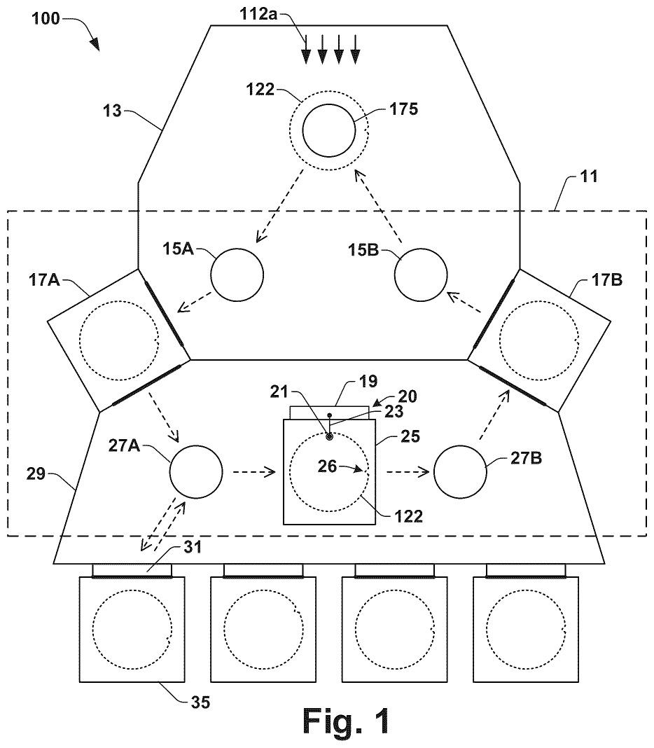

FIG. 1 illustrates an end station 100 for an ion implantation system in accordance with some embodiments of the present disclosure. The end station 100 includes a process chamber 13 and a front end module 29. The process chamber 13 is under vacuum and is where wafers 122 are implanted. The front end module 29 is kept at or near atmospheric pressure. An aligner 25, a first robot 27A, and a second robot 27B are disposed in the front end module 29. A workpiece handler 175, a third robot 15B, and a fourth robot 15A are disposed in the process chamber 13.

The first robot 27A reaches through one of the load ports 31 to draw a wafer 122 from a wafer carrier 35 and deposit it on the aligner 25. The wafer carriers 35 are multi-wafer transport modules such as wafer pods or wafer cassettes. In some embodiments, the wafer carriers 35 are front opening unified pods (FOUPs). An automated overhead transport or some other transport system may be used to move the wafer carriers 35 between the ion implantation system and other workstations in an integrated circuit (IC) fabrication plant.

The aligner 25 rotates the wafer 122 and sets its angular orientation with reference to an alignment mark 26 on the wafer 122. A wafer inspection system 20 inspects the wafer 122 while it is on the aligner 25. The wafer inspection system 20 includes an optical fiber 23, an output coupler 21, and components 19. The components 19 are mounted to a side of the aligner 25. The output coupler 21 may be held in a fixed position for inspecting the wafer 122 at an inspection point near its periphery.

A first load lock chamber 17B and a second load lock chamber 17A are positioned between the front end module 29 and the process chamber 13. The second robot 27B takes the wafer 122 from the aligner 25 and places it in the first load lock chamber 17B. After the first load lock chamber 17B has been evacuated, the third robot 15B takes the wafer 122 and sets it on the workpiece handler 175.

Ion implantation takes place while the wafer 122 is held by the workpiece handler 175. The workpiece handler 175 reorients the wafer 122 to align the wafer 122 on an implant plane. In some embodiments, the workpiece handler 175 sweeps the wafer 122 across the beam paths 112a in a slow scan direction while maintaining the wafer 122 on the implant plane. In some embodiments, the workpiece handler 175 remains stationary while the beam paths 112a sweep across the wafer 122 in fast scan and slow scan directions.

After ion implantation, the fourth robot 15A takes the wafer 122 from the workpiece handler 175 and places it in the second load lock chamber 17A. After the wafer 122 is brought to the pressure of the front end module 29, the fourth robot 15A withdraws the wafer 122 from the second load lock chamber 17A and returns it to one of the wafer carriers 35.

The components of the end station 100 that hold and transport the wafers 122 between the wafer carriers 35 and the workpiece handler 175 constitute a wafer transport system 11. In the wafer transport system 11, wafers 122 are brought into the process chamber 13 through the first load lock chamber 17B and are removed from the process chamber 13 through the second load lock chamber 17A, however, the load lock chambers 17A and 17B may each be used to transport wafers 122 in and out of the process chamber 13. In addition, a greater or lesser number of load lock chambers 17A and 17B may be provided.

The wafer transport system 11 uses the fourth robot 15A for transporting processed wafers 122 and the third robot 15B for transporting unprocessed wafer 122 within the process chamber 13, however, it will be appreciated that the fourth robot 15A may also transport unprocessed wafers 122 and the third robot 15B may also transport processed wafers 122. In addition, the wafer transport system 11 may have a greater or lesser number of robots in the process chamber 13.

The wafer transport system 11 uses the first robot 27A for transporting processed wafers 122 to wafer carriers 35 and for transporting unprocessed wafer 122 to the aligner 25. The wafer transport system 11 uses the second robot 27B for transporting wafers from the aligner to the first load lock chamber 17B. These functions may be otherwise distributed between the first robot 27A and the second robot 27B and may be distributed among a greater or lesser number of robots within the front end module 29. The number of load ports 31 through which the robots access wafer carriers 35 may also be varied without departing from the spirit and scope of the present disclosure.

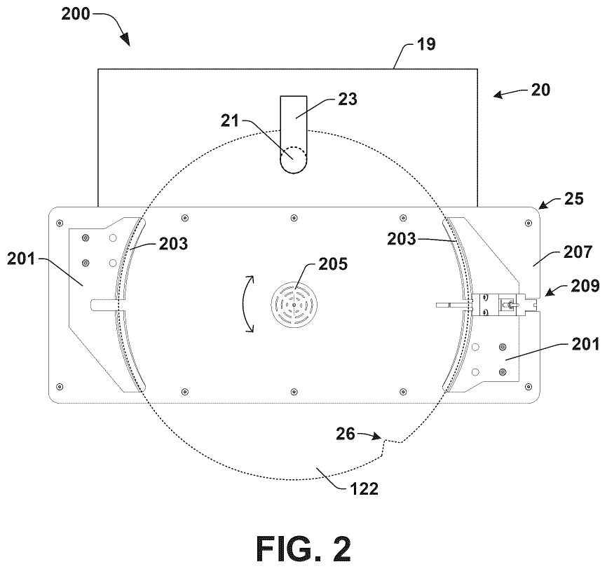

FIG. 2 provides a top view 200 illustrating an example of the aligner 25 using greater detail than is provided in FIG. 1. The aligner 25 includes a base 207, workpiece supports 201, a chuck 205, and an alignment mark detection device 209. The workpiece supports 201 may have arcuate recesses 203 configured to support a perimeter of the wafer 122. The chuck 205 may be rotatably mounted to the base 207 so that it is able to grip and rotate the wafer 122. The chuck 205 may be, for example, a vacuum chuck or an electrostatic chuck. The alignment mark detection device 209 may be an optical sensor or some other type of sensing device capable of detecting the alignment mark. The wafer inspection system 20 includes components 19 mounted to a side of the base 207 and an output coupler 21 held over the position occupied by the wafer 122 when held by the workpiece supports 201.

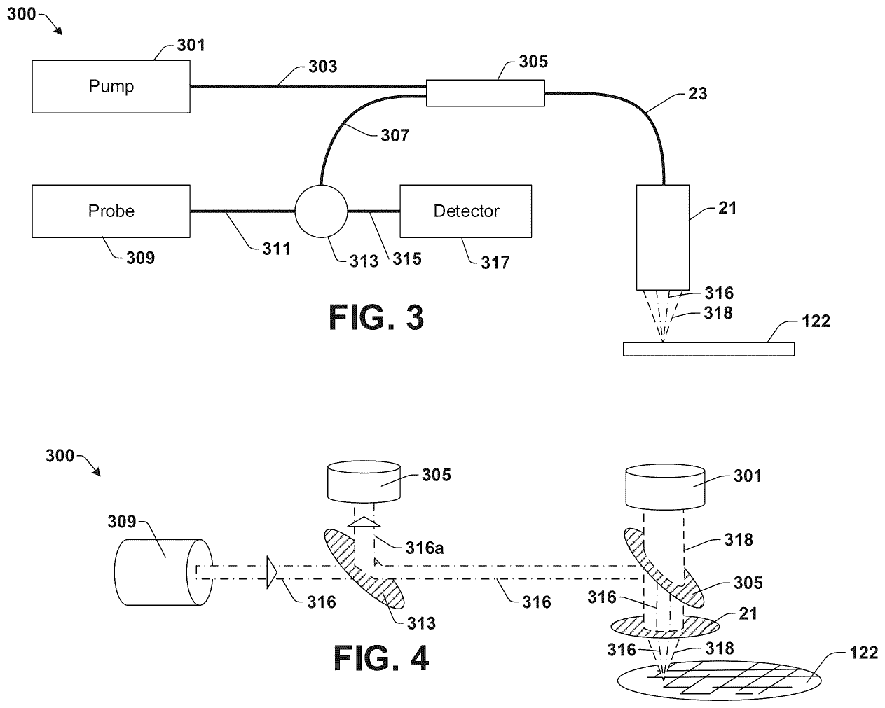

FIG. 3 illustrates an MOR system 300 which may provide the wafer inspection system 20. The MOR system 300 includes a pump laser 301, a wavelength coupler 305, a probe laser 309, a circulator 313, a photodetector 317, and an output coupler 21. Light 318 from the pump laser 301 is transmitted to the wavelength coupler 305 via an optical fiber 303 and from the wavelength coupler 305 to the output coupler 21 via the optical fiber 23. The output coupler 21 comprises a lens that focuses the light 318 onto the wafer 122. The light 318 stimulates a response at an inspection point on the wafer 122. The light 318 may be modulated to facilitate the detection of time-dependent responses in the wafer 122. In some embodiments, the modulation is amplitude modulation. Alternatively, or in addition, the light 318 may be frequency modulated.

Light 316 from the probe laser 309 is transmitted to the circulator 313 via an optical fiber 311, from the circulator 313 to the wavelength coupler 305 via an optical fiber 307, and from the wavelength coupler 305 to the output coupler 21 via the optical fiber 23. The lens in the output coupler 21 focuses the light 316 on the inspection point. Some of the light 316 reflects off the inspection point with an intensity that varies over time in a manner that is affected by the light 318 from the pump laser 301 in relationship to the properties of the wafer 122 at the inspection point. The reflected light 316 is collated by the lens in the output coupler 21, is transmitted from the output coupler 21 to the wavelength coupler 305 via the optical fiber 23, from the wavelength coupler 305 to the circulator 313 via the optical fiber 307, and from the circulator 313 to the photodetector 317 via an optical fiber 315. The optical fibers 23, 303, 307, 311, and 315, the wavelength coupler 305, the circulator 313, and the output coupler 21 comprise an optical circuit.

FIG. 4 provides an illustration of the MOR system 300 with the optical fibers removed and showing additional details for the wavelength coupler 305, the circulator 313, and the output coupler 21. As shown in FIG. 4, the circulator 313 may be a one-way mirror that transmits light 316 approaching from the direction of the probe laser 309 and reflects light approaching from the direction of the wavelength coupler 305. The wavelength coupler 305 may be a mirror that reflects the light 316 from the probe laser 309 and transmits the light 318 from the pump laser 301. The output coupler 21 may be a lens at the end of the optical fiber 23 (see FIG. 3).

The pump laser 301 has a wavelength suitable for creating electron-hole pairs in the wafer 122, which is a semiconductor. The semiconductor may be silicon (Si) or some other semiconductor material. In some embodiments, the wavelength is about 1 μm or less. In some embodiments, the frequency is in the blue part of the visible spectrum. In some embodiments, the pump laser 301 is an Argon laser and the light 318 has a wavelength of about 0.5 μm. In some embodiments, the pump laser 301 is a laser diode. The laser diode may provide light at a wavelength of about 405 nm, about 785 nm, or some other suitable wavelength. The pump laser 301 is powered by electronics that allow the amplitude of its output to be modulated. In some embodiments, the amplitude is modulated at a frequency in the range from about 1 kHz to about 40 kHz. Amplitude modulations in this range may be provided with inexpensive electronics. In some embodiments, the amplitude is modulated at a frequency in the range from about 40 kHz to about 1 MHz. Amplitude modulations in this range require more expensive electronics but may provide more information in a shorter period of time.

The variation in charge carrier concentration caused by the pump laser 301 affects the dielectric constant of the wafer 122 at the inspection point which in turn affects the refractive index. The pump laser 301 also causes local heating in the wafer 122 at the inspection point, which also affects refractive index. Both these effects cycle with the amplitude modulation. The cycles are out of phase which allows information from both types of effects to be extracted.

The probe laser 309 may operate with a constant amplitude so that amplitude modulation in the reflected light 316a at the photodetector 317 can be attributed to effects of the light 318 from the pump laser 301. The probe laser 309 may produce longer wavelength (lower energy) light than the pump laser 301. In some embodiments, the light 316 from the probe laser 309 has a wavelength of about 0.6 μm or greater. In some embodiments, the probe laser 309 is a Helium-Neon laser. In some embodiments, the probe laser 309 is a laser diode.

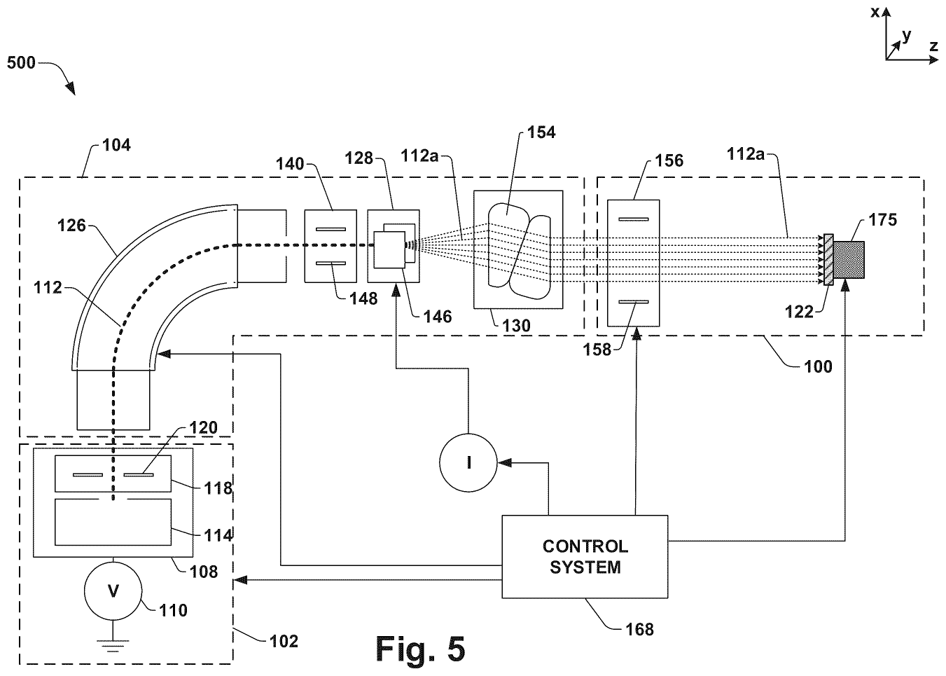

FIG. 5 illustrates an exemplary ion implantation system 500 that includes a terminal 102, a beamline 104, and the end station 100. The terminal 102 includes an ion source 108 powered by a high voltage power supply 110 that produces ions that are extracted and formed into an ion beam 112. The beamline 104 filters, shapes and steers the ion beam 112 to provide an x-direction sweep. The workpiece handler 175 in the end station 100 provides a y-direction sweep. Optionally, the beamline 104 steers the ion beam 112 to provide both the x-direction sweep and the y-direction sweep.

The ion source 108 may include an arc chamber 114 and an ion extraction assembly 118. The arc chamber 114 is supplied with a gas that includes the species to be implanted. Within the arc chamber 114, electrons are generated from an electron source. The electron source may be a filament or cathode that is heated with a current from the high voltage power supply 110 to induce thermionic emission of electrons. The electrons may be induced to arc and ionize some of the gas in the arc chamber 114 generating a plasma. A magnetic field may be provided to maintain the plasma in a swirl. The ions may be controllably extracted from the plasma and accelerated to a beamline energy by the ion extraction assembly 118. The ion extraction assembly 118 may include electrodes 120 that accelerate the extracted ions.

The beamline 104 may include a mass analyzer 126, a beam shaping and steering system 140, a scanning system 128, and a parallelizer 130. The mass analyzer 126 filters the ions based on charge-to-mass ratio so that after the mass analyzer 126 the ion beam 112 is a purified ion beam that includes only select ions. In the illustrated example, the mass analyzer 126 includes a bend through which the ions are deflected by a magnetic field. Ions having the wrong charge-to-mass ratio will be over-deflected or under-deflected so that only the ions having the desired charge-to-mass ratio continue down the beamline 104 from the mass analyzer 126.

The beam shaping and steering system 140 includes one or more electrical or magnetic lenses 148 that compress and steer the ion beam 112. In some embodiments, the beam shaping and steering system 140 includes a first quadrupole magnet that squeezes the ion beam 112 in the x-direction (an x-quad) and a second quadrupole magnet that squeezes the ion beam 112 in the y-direction (an y-quad). The y-direction is into the page of FIG. 5.

The scanning system 128 steers the ion beam 112 so that a beam path 112a for the ion beam 112 sweeps across the x-direction (fast scan direction). The scanning system 128 may include plates 146. The plates 146 may steer the ion beam 112 either electrically or magnetically. In some embodiments, the scanning system 128 sweeps the beam path 112a cyclically at a rate of about 1 kHz or more. The sweeps in the y-direction (slow scan direction) are at a slower rate, e.g., about 100 Hz or less.

The scanned ion beam 112 may be passed through a parallelizer 130. In the illustrated example, the parallelizer 130 includes two dipole magnets 154. The two dipole magnets 154 may be substantially trapezoidal and oriented to mirror one another and bend the beam paths 112a into s-shapes. The parallelizer 130 has the effect of making all the beam paths 112a substantially parallel.

The ion beam 112 is received by the end station 100. Optionally, a deceleration stage 156 is provided within the end station 100 downstream from the parallelizer 130. The deceleration stage 156 may include one or more electrodes 158 that slow the ion beam 112 and focus the ion beam 112 into a converging stream. In some embodiments, the beamline 104 is maintained at a first potential and the deceleration stage 156 is maintained at a second potential, wherein the first potential corresponds to the beamline energy and the second potential corresponds to the implantation energy. The apparatus that focuses the ion beam 112 may be integral with the apparatus that slows the ion beam 112. In some embodiments, the deceleration stage 156 includes an Einzel lens.

A control system 168 is provided to control, communicate with, and/or adjust the ion source 108, the mass analyzer 126, the scanning system 128, the deceleration stage 156, and the workpiece handler 175. The control system 168 may also control the wafer transport system 11 and the wafer inspection system 20 (see FIG. 1) or may communicate with one or more comparable control systems provided for the wafer transport system 11 and the wafer inspection system 20. The control system 168 may comprise a computer including a central processing unit and a memory system programmed with instructions for operating these and other components of the ion implantation system 500. For example, the control system 168 may determine the rate of ion production in the ion source 108, the beamline energy, the fast scan rate, the implantation energy, and the slow scan rate. The control system 168 may receive a set of operating parameters constituting a recipe through which an operator may direct the ion implantation system 500 to conduct a specific and reproducible ion implantation operation. The control system 168 may operate the wafer transport system 11 to maximize throughput and may analyze and, in some cases, respond to data from the wafer inspection system 20.

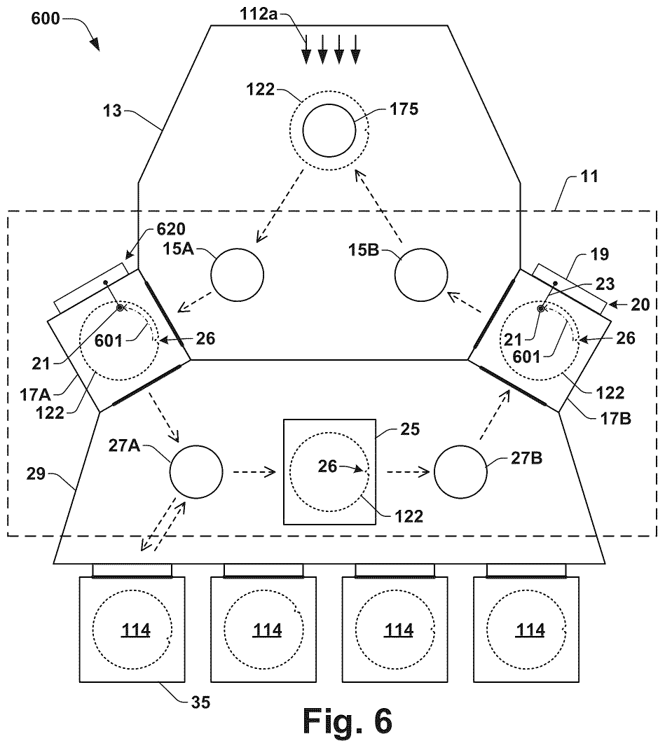

FIG. 6 illustrates an end station 600 according to another embodiment. The end station 600 is like the end station 100 of FIG. 1 except that in the end station 600 the wafer inspection system 20 is configured to inspect the wafer 122 while the wafer 122 is in the first load lock chamber 17B. The optical fiber 23 may pass through a vacuum-sealed port in the top of the first load lock chamber 17B so that the output coupler 21 is within the first load lock chamber 17B and the components 19 are outside the first load lock chamber 17B. In some embodiments, the components 19 are attached to a side of the first load lock chamber 17B.

As shown in FIG. 6, the end station 600 may include a second wafer inspection system 620 installed in the second load lock chamber 17A. The second wafer inspection system 620 is provided to inspect the wafer 122 after ion implantation. The output coupler 21 of the second wafer inspection system 620 may be positioned to inspect the wafer 122 at the same point as does the wafer inspection system 20 to facilitate comparison between data from the second wafer inspection system 620 and data from the wafer inspection system 20. That point is an angular distance 601 from the alignment mark 26.

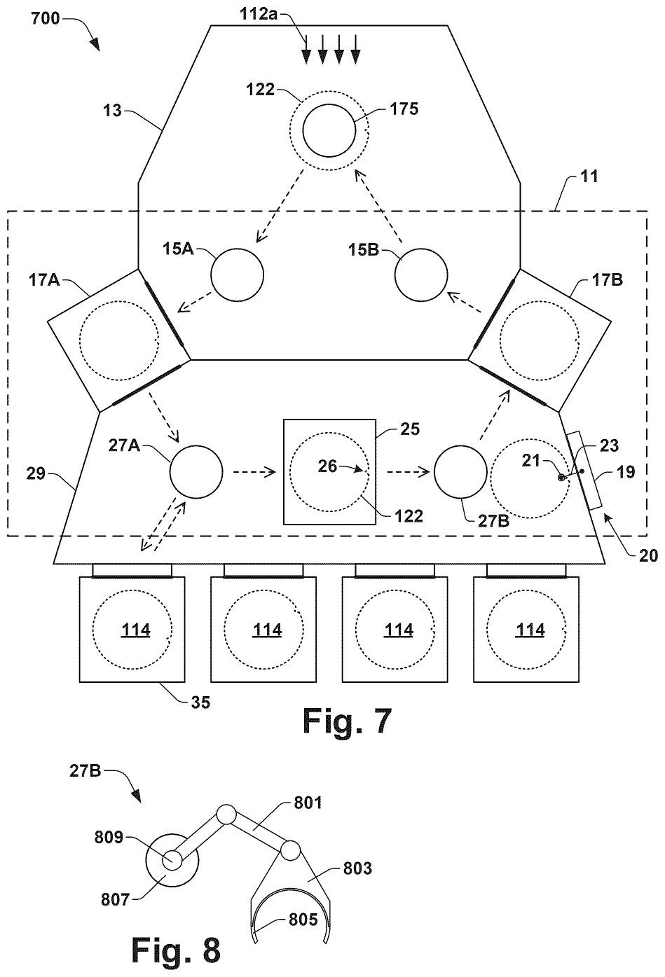

FIG. 7 illustrates an end station 700 according to another embodiment. The end station 700 is like the end station 100 of FIG. 1 except that in the end station 700 the wafer inspection system 20 is configured to inspect the wafer 122 while the wafer 122 is being held by the second robot 27B or some other robot of the wafer transport system 11. In some embodiments, the components 19 are within the front end module 29. In some embodiments, the components 19 are outside the front end module 29 and the optical fiber 23 passes through a wall of the front end module 29 as shown in FIG. 7.

The robot 27B may have any suitable structure. FIG. 8 illustrates a configuration for the second robot 27B in accordance with some embodiments. As shown in FIG. 8, the second robot 27B includes a base 807, three pivoting joints 809, two arms 801, and an effector 803. The effector 803 may have an arcuate recess 805 for supporting the wafer 122 at its perimeter. In some embodiments, the arms 801 may be raised or lowered with respect to the base 807. These features, or their like, allow the second robot 27B to place the wafer 122 beneath the output coupler 21 (see FIG. 7) so that the wafer 122 may be inspected while it is held by the effector 803. Instead of the mechanical effector 803, the second robot 27B may have an electrostatic chuck, a vacuum chuck, or some other device that holds or supports the wafer 122 as it is moved by the second robot 27B. The robots 27A, 15A, and 15B may have similar configurations.

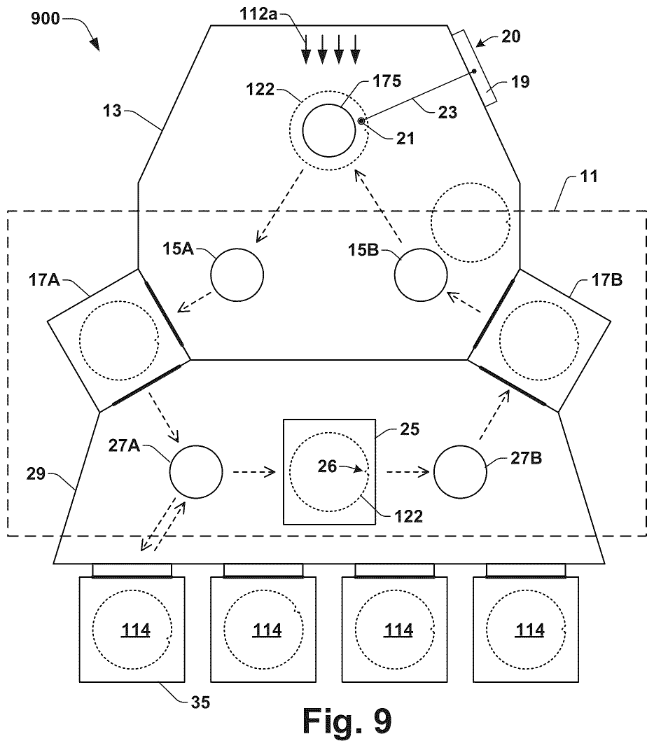

FIG. 9 illustrates an end station 900 according to another embodiment. The end station 900 is like the end station 100 of FIG. 1 except that in the end station 900 the wafer inspection system 20 is configured to inspect the wafer 122 while the wafer 122 is being held by the workpiece handler 175. In some embodiments, the components 19 are within the process chamber 13. In some embodiments, the components 19 are outside the process chamber 13 and the optical fiber 23 passes through a wall of the process chamber 13 as shown in FIG. 9.

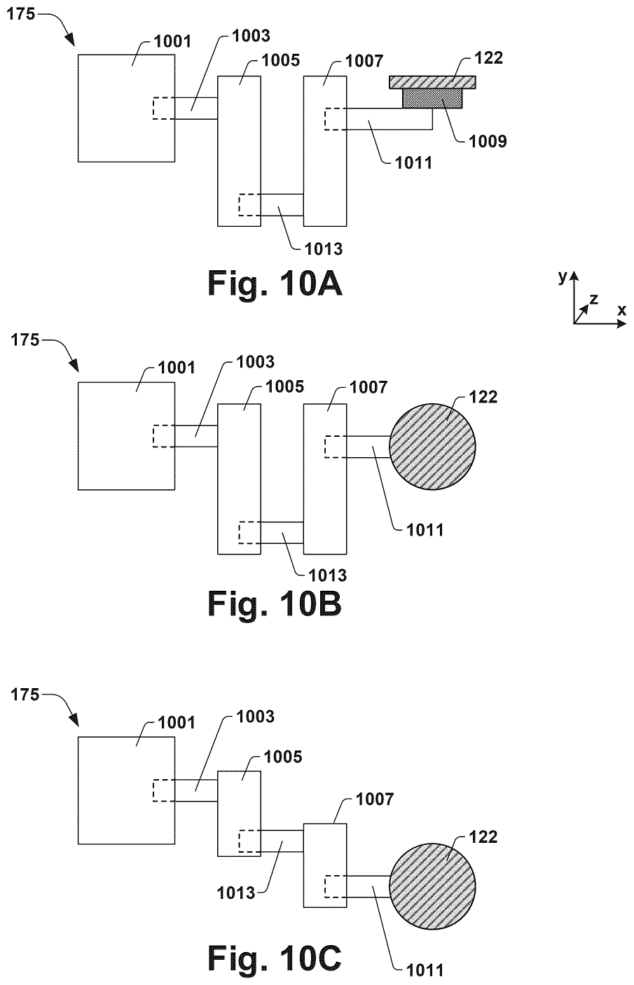

FIGS. 10A-10C illustrate an example of the workpiece handler 175 in accordance with some embodiments. As shown in FIG. 10A-10C, the workpiece handler 175 includes a base 1001, an arm 1005, an arm 1007, and a chuck 1009. The chuck 1009 may be a mechanical chuck, an electrostatic chuck, a vacuum chuck, or some other type of chuck suitable for holding the wafer 122 during ion implantation. The chuck 1009 is pivotally connected to an arm 1007 through a joint 1011. The arm 1007 is pivotally connected to the arm 1005 through a joint 1013. The arm 1005 is pivotally connected to a base 1001 through a joint 1003.

As shown in FIG. 10A, the wafer 122 may be facing in the y-direction when it is received by the third robot 15B (see FIG. 9). The y-direction may be a vertical direction. The wafer 122 may be inspected while having this orientation.

As shown in FIG. 10B, the wafer 122 may be rotated about the joint 1011 so that the wafer 122 is oriented in an implant plane (compare FIG. 5). The implant plane is selected according to a desired orientation of the wafer 122 with respect to the beam paths 112a. The beam paths 112a are in the z-direction and in the examples shown in FIGS. 10B and 5, the implant plane is set so as to be perpendicular to the z-direction. Optionally, the implant plane is angled with respect to the z-direction. As shown in FIG. 10C, by coordinated rotation about the joints 1003, 1011, and 1013, the wafer 122 may be swept in the y-direction while keeping the wafer 122 oriented on the implant plane.

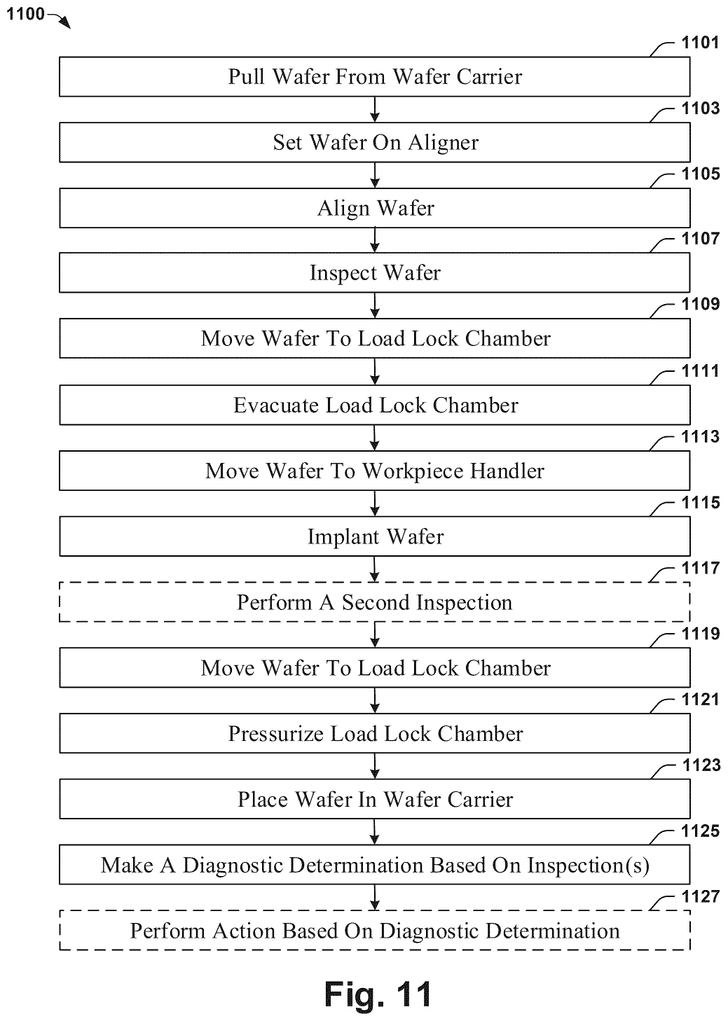

FIG. 11 provides a flow chart of a method 1100 that may be implemented using the ion implantation system 500 together with one of the end stations 100, 600, 700, or 900. While the method 1100 is illustrated and described below as a series of acts or events, it will be appreciated that the illustrated ordering of such acts or events is not to be interpreted in a limiting sense. For example, some acts may occur in different orders and/or concurrently with other acts or events apart from those illustrated and/or described herein. In addition, not all illustrated acts may be required to implement one or more aspects or embodiments of the description herein.

The method 1100 may begin with act 1101, removing a wafer from a wafer carrier. FIG. 1 provides an example in which the first robot 27A in the front end module 29 performs this action. Alternatively, the wafer carrier 35 is transported into the process chamber 13 and this action takes place in the process chamber 13.

Act 1103 is placing the wafer on an aligner. Act 1105 is using the aligner to set the wafer alignment.

Act 1107 is inspecting the wafer. In some embodiments, the inspection is performed through an optical fiber. In some embodiment, the inspection comprises MOR inspection. In some embodiment, the inspection is performed while the wafer is on the aligner. FIG. 1 provides an example of an end station that enables this embodiment. In some embodiments, this wafer is rotated on the aligner and the inspection is repeated. In some embodiment, the inspection is performed after the wafer has been aligned. In some embodiments, the inspection is performed while the wafer is a load lock chamber. FIG. 6 provides an example of an end station that enables this embodiment. In some embodiments, the inspection is performed while the wafer is in the grip of a robot. FIG. 7 provides an example of an end station that enables this embodiment. In some embodiments, the inspection is performed while the wafer is held by the workpiece handler. FIG. 9 provides an example of an end station that enables this embodiment.

Act 1109 is moving the wafer to a load lock chamber. Act 1111 is evacuating the load lock chamber. Act 1113 is moving the wafer from the load lock chamber to a workpiece handler. Act 1115 is performing an ion implantation on the wafer while the wafer is held by the workpiece handler.

Act 1117 is an optional step of performing a second inspection of the wafer after the ion implantation. This second inspection may take place in the process chamber, in the load lock chamber, or in the front end module.

Act 1119 is moving the wafer from the workpiece handler to a load lock chamber. Act 1121 is pressurizing the load lock chamber. Act 1123 is moving the wafer from the load lock chamber to a wafer carrier. The wafer carrier may be the same wafer carrier from which the wafer was taken or a different wafer carrier.

Act 1125 is making a diagnostic determination based on the data obtained using Act 1107, which is prior to the ion implantation of Act 1115. In some embodiments, the diagnostic determination pertains to a state of the wafer prior to ion implantation. In some embodiments, the diagnostic determination comprises comparing the data to reference data obtained previously by inspecting one or more other wafers. In some embodiments, the diagnostic determination comprises a determination of a free carrier concentration at the inspection point. For example, the parameters needed to apply the Drude model for determining free carrier concentration may be obtained from MOR data.

In some embodiments, the diagnostic determination comprises an assessment of a degree of amorphism. For example, MOR data provides peaks corresponding to band gap energies. If the material has a highly order crystal structure, these peaks will be sharp. As amorphism increases, these peaks become more diffuse.

In some embodiments, the diagnostic determination further comprises using data obtained using Act 1117, which is a second inspection that is performed after the ion implantation of Act 1115. In that case, the diagnostic determination may assess the effectiveness of Act 1117 in obtaining a desired result.

Act 1127 is an optional act of performing some action based on the diagnostic determination. In some embodiments, Act 1127 is modifying a parameter of the ion implantation of Act 1115. For example, the dose of the ion implantation may be increased or decreased based on the results of the inspection. In some embodiments, the modification is applied to the wafer that was inspected. Alternatively, or in addition, the modification may be applied to ion implantations carried out on other wafers. In some embodiments, a remedial action other than modifying the ion implantation of Act 1115 is made for the inspected wafer based on the diagnostic determination. In some embodiments, the ion implantation of Act 1115 is bypassed for the inspected wafer based on the diagnostic determination. For example, the ion implantation may be postponed until after the wafer has been cleaned or returned to an upstream process that was not completed correctly. The upstream process may be another ion implantation process used for one of doping or damage engineering. In some embodiments, a wafer is selectively discarded based on the diagnostic determination.

In some embodiments, a process upstream from the ion implantation system is modified based on the diagnostic determination. For example, the conditions for an earlier ion implantation process for either doping or damage engineering may be modified based on the diagnostic determination.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure. In addition, while a particular feature may have been disclosed with respect to only one of several embodiments, such feature may be combined with one or more other features of the other embodiments as may be desired or advantageous for a given application.

What is claimed is:

1. An ion implantation system, comprising:
an ion source configured to generate an ion beam;
a beamline configured to guide the ion beam;
an end station positioned to receive the ion beam;
a workpiece handler in the end station, wherein the workpiece handler is configured to move a wafer into a path of the ion beam while holding the wafer on a chuck;
a wafer transport system within the end station, wherein the wafer transport system is configured to move the wafer from a wafer carrier to the workpiece handler; and
a wafer inspection system comprising an optical fiber and a lens, wherein the wafer inspection system is configured to inspect the wafer through the optical fiber while the wafer is in transit from the wafer carrier to the path of the ion beam.

2. The ion implantation system of claim 1, wherein the wafer inspection system is positioned to inspect the wafer while the wafer is being handled by the wafer transport system.

3. The ion implantation system of claim 2, wherein the wafer inspection system is positioned to inspect the wafer while the wafer is in a load lock chamber.

4. The ion implantation system of claim 2, wherein the wafer inspection system is positioned to inspect the wafer while the wafer is on an aligner.

5. The ion implantation system of claim 4, wherein a component of the wafer inspection system is mounted on the aligner.

6. The ion implantation system of claim 1, wherein the wafer inspection system is positioned to inspect the wafer while the wafer is held by the workpiece handler.

7. The ion implantation system of claim 1, wherein the end station comprises a vacuum chamber and the wafer inspection system is positioned to inspect the wafer while the wafer is in the vacuum chamber.

8. The ion implantation system of claim 1, wherein the wafer inspection system comprises a modulated optical reflectance system.

9. The ion implantation system of claim 1, wherein the wafer carrier is a front opening unified pod.

10. The ion implantation system of claim 1, wherein the wafer inspection system further comprises, a first laser, a second laser, and a wavelength coupler, wherein the wavelength coupler is configured to guide light from both the first laser and the second laser to the optical fiber.

11. An ion implantation system, comprising:
an ion source configured to generate an ion beam;
a beamline configured to guide the ion beam;
an end station positioned to receive the ion beam;
a workpiece handler in the end station, wherein the workpiece handler is configured to hold a wafer in a path of the ion beam;
a wafer transport system comprising an aligner, wherein the wafer transport system is configured to set an orientation of the wafer using the aligner and to transfer the wafer from the aligner to the workpiece handler; and
a wafer inspection system comprising an optical fiber, wherein the wafer inspection system is positioned to inspect the wafer through the optical fiber while the wafer is on the aligner.

12. The ion implantation system of claim 11, wherein a component of the wafer inspection system is mounted on the aligner.

13. A method of operating an ion implantation system, the method comprising:
picking a wafer from a wafer carrier using a wafer transport system that is part of an ion implantation system;
placing the wafer on an aligner;
setting an alignment of the wafer;
performing a first inspection on the wafer to obtain first inspection data;
transporting the wafer from the aligner to a workpiece handler of the ion implantation system; and
after performing the first inspection and while the wafer is held by the workpiece handler, performing an ion implantation on the wafer.

14. The method of claim 13, wherein the first inspection takes place while the wafer is on the aligner.

15. The method of claim 14, wherein the first inspection takes place while the wafer is on the aligner and after setting the alignment of the wafer.

16. The method of claim 14, wherein the first inspection comprises inspecting the wafer while the wafer is at a first orientation and the method further comprises performing a second inspection on the wafer while the wafer at a second orientation.

17. The method of claim 13, further comprising:
performing a second inspection of the wafer after the ion implantation to obtain second inspection data; and
providing a diagnostic determination based on a comparison between the first inspection data and the second inspection data.

18. The method of claim 13, wherein performing the first inspection comprises modulated optical reflectance spectroscopy.

19. The method of claim 18, wherein performing the modulated optical reflectance spectroscopy comprises using a wavelength coupler to combine an output of a pump laser and an output of a probe laser into an optical fiber and focusing the output of the pump laser and the output of the probe laser on an inspection point on the wafer.

20. The method of claim 13, wherein the ion implantation is carried out with a condition set based on the first inspection data.
